(19) United States
(12) Patent Application Publication
(10) Pub. No.: US 2018/0028343 A1
(43) Pub. Date: Feb. 1, 2018

(54) Apparatus, System and Method for Providing Back Support

(71) Applicants: Gabriel McGee, Blue Island, IL (US); Theodore McGee, Blue Island, IL (US)

(72) Inventors: Gabriel McGee, Blue Island, IL (US); Theodore McGee, Blue Island, IL (US)

(21) Appl. No.: 15/220,099

(22) Filed: Jul. 26, 2016

(51) Int. Cl. A61F 5/02

(52) U.S. Cl. CPC A61F 5/028

(57) ABSTRACT

A belt for providing lower back support to the wearer of the belt has a first section and a second section that is substantially symmetrical to and positioned opposite the first section. The first section connects to the second section at a rear connection region. The first section has a first fastener panel, and the second section has a second fastener panel corresponding to and positioned to attach to the first fastener panel to secure the belt around a lower back area of a wearer of the belt. The first section and the second section have a reinforcement plate at a rear connection region of the belt. An adhesive strip extends across the first section and the second section from the rear connection region. A cord interwoven through the reinforcement plate is pulled to secure the first section to the second section and to tighten the belt around the wearer.

### TECHNICAL FIELD

The present invention generally relates to an apparatus, a system and a method for compressing the abdomen of a wearer to provide spinal, lower back and/or lumbar support to assist lifting objects, such as those encountered while moving furniture. More particularly, the invention relates to a multi-piece adjustable back support belt made from breathable mesh stretched across a canvas perimeter. The back support belt has reinforcement plates that tighten to secure the belt around a wearer by pulling a cord threaded through the plates.

### BACKGROUND OF THE INVENTION

Movers, luggage carriers, food transporters and/or other laborers often bend to pick up items, such as furniture and/or cargo, for example, to transport the items to a desired final position and/or destination, such as a house and/or a cargo truck, for example. The act of retrieving the items may require the laborer to bend at a peculiar and/or an unnatural angle, thus potentially contributing to lower back pain and/or discomfort of the laborer. Even the most skilled laborer may experience back pain and/or discomfort over time after performing routine, repetitive tasks that are strenuous and/or taxing on the body. Further, other factors may contribute toward back discomfort and/or pain experienced by such laborers, such as aging, muscle deterioration around the spine and/or wearing heavy protective clothing, i.e. where the weight of such clothing places stress upon the back of the wearer, for example.

Various devices and methods have been used to address body pain and/or injury issues associated with repetitive and/or strenuous heavy lifting. Specific lifting techniques may encourage the laborer to use his or her knees to provide force to grasp and/or to lift a heavy object, rather than bending downwards at the waist where doing so may strain the back of the laborer. However, such measures may be insufficient to protect the back of the laborer who is routinely required to perform demanding heavy lifting tasks.

Accordingly, back support devices, such as, for example, back support belts have been developed to address injuries associated with the repetitive lifting of heavy objects. For instance, U.S. Pat. No. 9,095,418 to Cardinali et al. discloses an orthosis having a tensioning device with an anti-twist mechanism which includes members with a reduced degree of pliability to resist twisting of the orthosis. U.S. Pat. No. 9,066,792 to Garth et al. discloses a back brace to fit a wearer in a multitude of different configurations. The back brace has a lumbar support split into upper and lower sections that may be connected to a flexible joint allowing for the lumbar support to bend toward the spine of the user. U.S. Pat. No. 8,956,315 to Garth et al. discloses a double pull body brace having a panel that engages around the torso of a wearer and overlaps at the front of the panel. U.S. Pat. No. 8,945,033 to Guldalian discloses a segmented lumbosacral orthosis with two side members conjoined by a cord threaded through an array of seamless tubes. Each tube swivels from an axis point near to an end of each tube. U.S. Pat. No. 8,864,695 to Thornton discloses an adjustable brace apparatus with a pair of elongate wings configured for wrapping about and substantially conforming to a body of a user.

Further, U.S. Pat. No. 8,409,122 to Cropper et al. relates to a lumbar orthosis that has opposing and mating front attachment panels and an adjustable, rigid lumbar compression piece configured for positioning at the rear of a wearer. Pulling of a cord causes the brace both circumferentially to tighten and to concentrate compression and pressure of the rigid lumbar compression piece. U.S. Pat. No. 7,727,172 to Wang discloses a back brace that has a first brace member with a first connector, a second brace member with a second connector, and a coupler at opposing sides thereof. The coupler is detachably connectable to the first connector. U.S. Pat. No. 7,306,571 to Schwenn et al. discloses an orthosis having a first body member to conform to a portion of a patient's body, and a second body member that conforms to another portion of a patient's body. Moreover, U.S. Pat. Nos. 7,118,543; 7,083,585; 6,342,044; 3,945,041 and U.S. Patent Publication Nos. 2014/0142485; 2009/0192425; 2007/0073204 and 2001/0020144 generally relate to back support belts and/or other similar orthopedic lumbar support devices.

Devices for providing lower back and/or lumbar support to a wearer of the device by wrapping an adjustable belt around the waist of a wearer are known. However, such devices may be suitable for particular lifting situations and/or back positions rather than offering a solution suitable for a variety of circumstances for a given intended lift load, for example. Moreover, larger, heavier and/or otherwise more substantial belts may present a difficulty in terms of ease of storage and/or portability, thus further limiting their use and/or utility. Accordingly, a need exists for a back support belt that may be stored, transported and/or configured for use. The back support belt may have multiple pieces for positioning and/or assembling by attachment of corresponding fastener panels as desired by a wearer. Further, the wearer may tighten the back support belt by pulling a cord threaded through rigid reinforcement pieces on the back support belt.

### SUMMARY OF THE INVENTION

The present invention generally relates to an apparatus, a system and a method for providing lower back and/or lumbar support to assist lifting objects, such as those encountered while moving furniture. More particularly, the invention relates to a multi-piece adjustable back support belt made from breathable mesh stretched across a canvas perimeter. The back support belt has reinforcement plates that may be tightened to further secure the belt around a wearer by pulling a cord threaded through the plates.

The invention provides an apparatus having a belt with a first section and a second section positioned opposite to the first section. The first section may connect to the second section at a rear connection region. A reinforcement plate may be attached to the first section and the second section at the rear connection region. An adhesive strip may extend lengthwise across the first section and the second section. A first fastener panel may be attached to the first section and a second fastener panel may be attached to the second section. The second fastener panel may be positioned to correspond with and attach to the first fastener to secure the belt around a lower back area of a wearer of the belt. A cord may be interwoven through the reinforcement plate and may be pulled to secure the first section to the second section.

Additionally, the invention provides a system having a belt having a first side and a second side positioned opposite to the first side. The first side and the second side may have a perimeter with a mesh stretched across the perimeter. A plate may be attached to the mesh of the first side and the second side. The plate may be positioned adjacent to the lower back of a wearer of the belt to support the wearer during movement. A strip may be attached to the mesh where the strip may extend lengthwise across the belt. A cord may be threaded through the plate where pulling of the cord may compress the belt to further secure the belt on the wearer. The cord may attach to the strip to retain the belt in a fixed position.

Further, the invention provides a method for wrapping a belt around a lower back area of the wearer. The belt may have a first side section and a second side section substantially identical to and positioned adjacent to the first side section. A fastener panel on the first side section may be attached to a corresponding fastener on the second side section to secure the belt around the lower back area of the wearer. A cord threaded through the first side section and the second side section may be pulled to tighten the belt around the wearer. The cord may be fixed onto an adhesive strip that may extend across the first side section and the second side section to secure the belt in a fixed position on the wearer.

Accordingly, it is an advantage of the present invention to provide an apparatus, a system and a method that may be easily transported and/or configured to provide lower back and/or lumbar support to assist in lifting objects, such as those encountered while moving furniture.

Another advantage of the present invention is to provide a belt with a first section and a second section that may extend from a rear connection region of the belt to wrap around a lower back area of a wearer of the belt to secure the spine region of a wearer of the belt.

Yet another advantage of the present invention is to provide a belt that accommodates convenient fastening of two corresponding fastener panels, where each fastener panel is located on an opposing side of the belt.

A further advantage of the present invention is to provide a belt with a bungee cord that may be pulled by a wearer of a belt to secure the belt in a tightened position around the wearer.

Additional features and advantages of the present invention are described in, and will be apparent from, the detailed description of the presently preferred embodiments and from the drawings.

### BRIEF DESCRIPTION OF THE DRAWINGS

Exemplary embodiments of the present embodiments are described herein with reference to the following drawings.

FIG. 1 illustrates a perspective view of a support belt with a cord interlaced through a support portion in accordance with an embodiment of the invention.

FIG. 2 illustrates a perspective view of a support belt with a fastener strap connected to an adjustment cord in accordance with an embodiment of the invention.

FIG. 3 illustrates a perspective view of inward-facing surfaces of two connected mesh panels of the support belt in accordance with an embodiment of the invention.

FIG. 4 illustrates a top view of the support belt with one mesh panel folded and placed on another mesh panel in accordance with an embodiment of the invention.

FIG. 5 illustrates a top view of the back support belt with one mesh panel folded and placed on another mesh panel in accordance with an embodiment of the invention.

FIG. 6 illustrates a top view of the back support belt folded to a reduced size in accordance with an embodiment of the invention.

FIG. 7 illustrates a detailed view of a fastener strap connected to an elastic adjustment cord extending from the back support belt in accordance with an embodiment of the invention.

FIG. 8 illustrates a detailed view of a fastener attachment region on an inward-facing surface of a mesh panel in accordance with an embodiment of the invention.

FIG. 9 illustrates a front view of the back support belt worn by a wearer in accordance with an embodiment of the invention.

FIG. 10 illustrates a front view of the back support belt worn by a wearer in accordance with an embodiment of the invention.

### DETAILED DESCRIPTION OF THE PRESENTLY PREFERRED EMBODIMENTS

In the following description, numerous details are set forth to provide an understanding of the present invention. Further, numerous variations or modifications from the described embodiments may be possible.

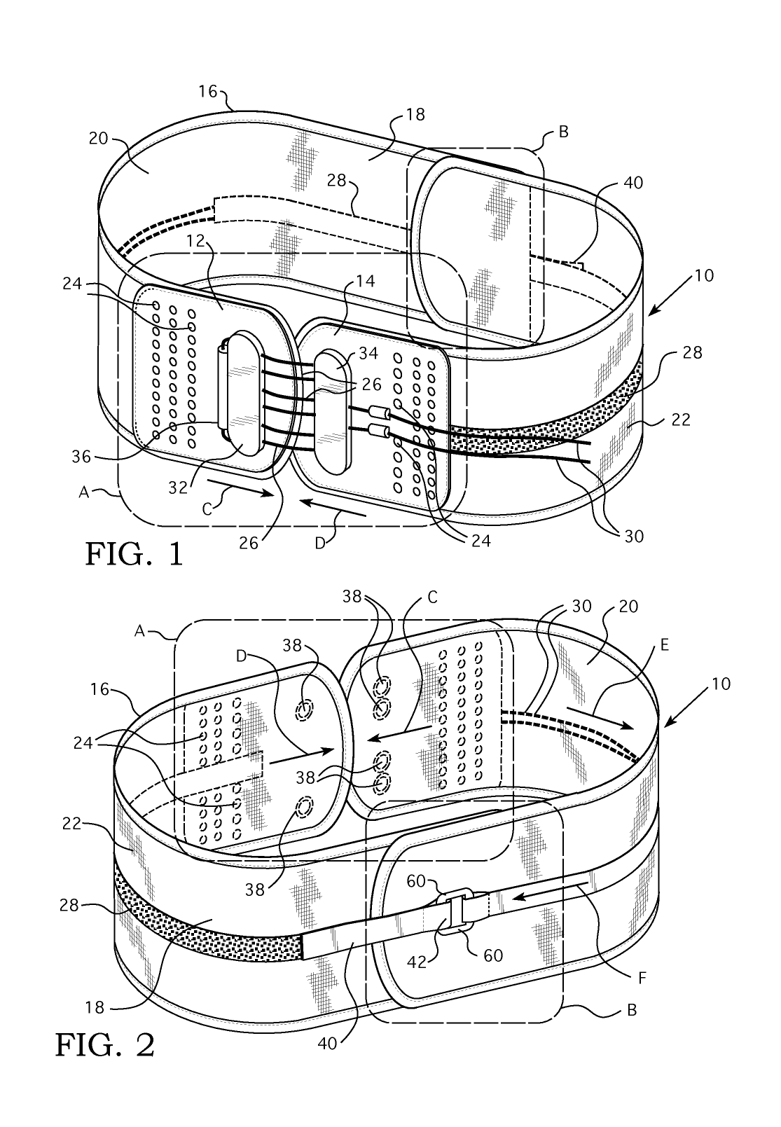

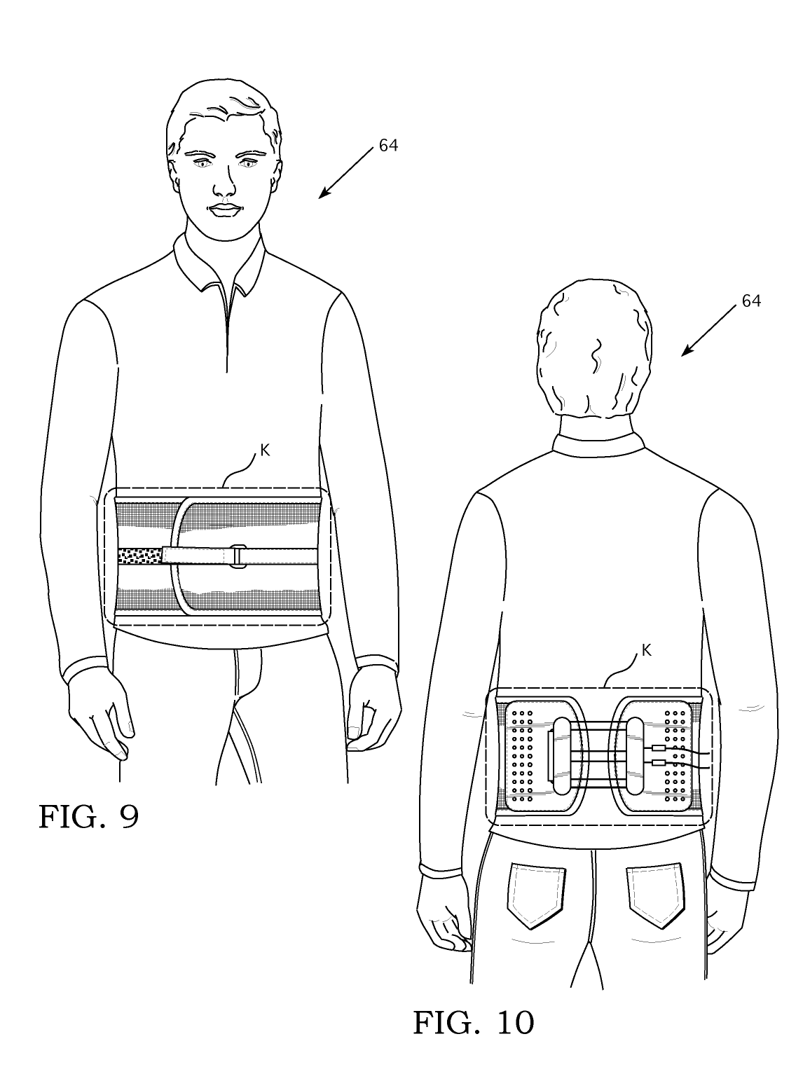

Referring to FIG. 1, a support belt 10 is shown. In an embodiment, the support belt 10 may be referred to as a back and/or lumbar support belt and/or brace, a lumbosacral orthosis and/or any other device and/or belt that may be worn generally around the waist, midsection and/or lower back area K of a wearer 64 as shown in FIGS. 9 and 10, for example. A circumference of the support belt 10 may be adjusted and/or reduced as necessary to accommodate the girth of the wearer 64. The support belt 10 may provide reinforcing support to the lower back area K of the wearer 64 during, for example, physically taxing and/or otherwise strenuous activity, such as, but not limited to, bending, lifting and/or moving heavy items, such as furniture and/or cargo.

The support belt 10 may compress against the spine of the wearer 64 at, for example, the lower back area K, to prevent unwanted and/or sudden movement of the spine that may hurt and/or otherwise harm the wearer 64. Further, in an embodiment, the wearer 64 may adjust the tension and/or compression of the support belt 10 around the lower back area K of the wearer 64 to accommodate various tasks. Each task may demand and/or be suited for a particular level and/or degree of compression of the support belt 10. For example, more strenuous activity over a short duration may demand a higher degree of compression of the support belt 10 around the waist and/or girth of the wearer 64. Likewise, less strenuous activity taking place over a longer period of time may require only a relatively loose compression of the support belt 10 around the waist of the wearer 64. Moreover, the support belt 10 may be made in conformance with specific dimensions, such as height, weight and/or thickness, for example. Heavier ones of the support belt 10 may be better suited for more demanding tasks undertaken by the wearer 10. Similarly, lighter ones of the support belt 10 may be better suited for less demanding tasks.

The support belt 10 may be defined by a perimeter 16 made from a pliable and/or resilient fabric material, such as, for example, canvas, denim, cotton and/or a synthetic fiber blend. A mesh 18 that may be made from interwoven fibers may stretch across the perimeter 16 to form a first side piece 20 that may connect with and/or otherwise attach to a second side piece 22. The first side piece 20 may be substantially symmetrical to the second side piece 22, at both a rear connection region A and a front connection region B as shown in FIG. 1. In an embodiment, the first side piece 20 and/or the second side piece 22 may be referred to as a “section” of the support belt 10. Both the first side piece 20 and the second side piece 22 may have an adhesive strip 28 that may be, for example, attached to and/or stitched onto the mesh 18. In an embodiment, the adhesive strip 28 may engage with a tab 40 which may be connected to and/or otherwise extend from a cord 30, as shown in FIG. 2, for example, to secure the belt 10 in a fixed position.

The cord 30 may be made from an elastic material that may be configured to reversibly deform upon receiving a force, such as being stretched and/or pulled in a given direction and, in an embodiment, may be referred to as a “bungee,” “bungie” and/or as a shock cord. Resilient fibers within the cord 30 may “snap-back” and/or otherwise return to an original, resting position thus allowing for the cord 30 to occupy limited volume when not use. Further, such resilient fibers may be stretched as necessary to attach to the adhesive strip 28 to secure the support belt 10 around the lower back area K of the wearer, as shown in FIGS. 9 and 10, for example.

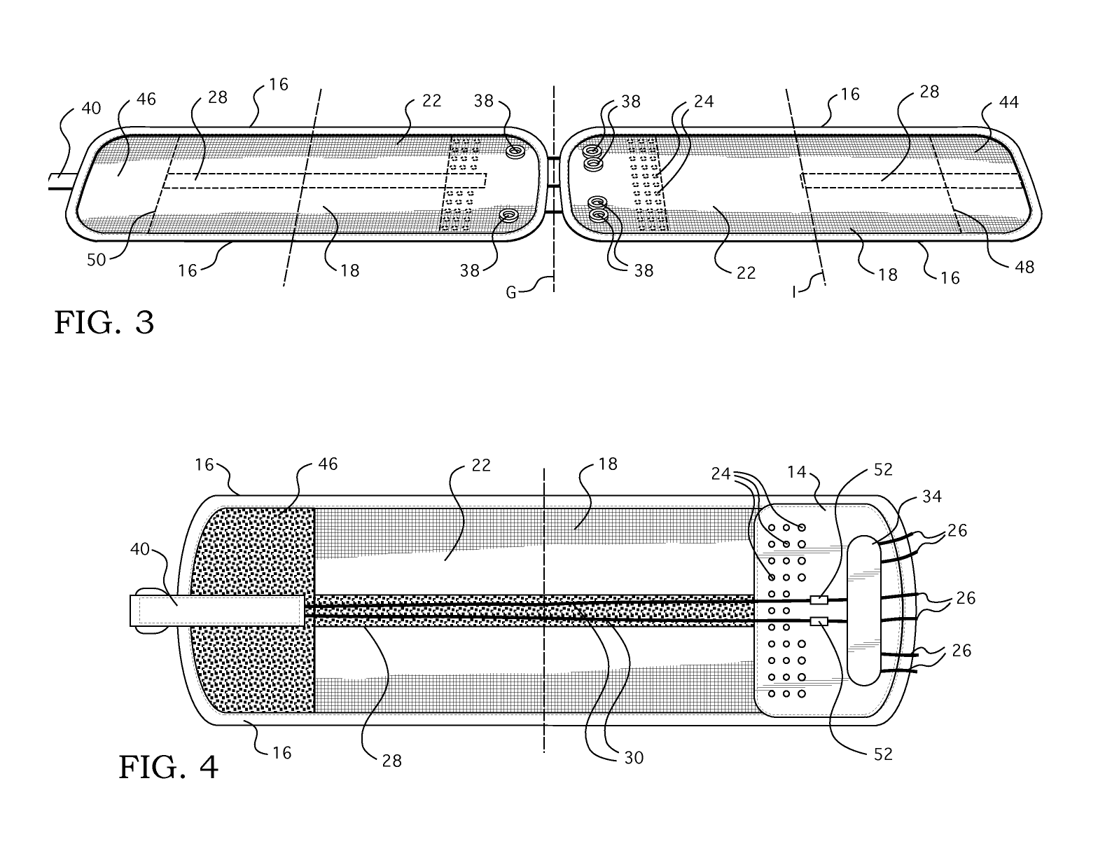

In an embodiment, the tab 40, the adhesive strip 28, a first fastener panel 44 and/or a second fastener panel 46, as shown in FIG. 3, for example, may be Velcro®, a registered trademark of Velcro USA, Inc., a Delaware corporation, and/or otherwise similar to Velcro® in form and/or function. The tab 40 may be extended and/or pulled by the wearer 64 to wrap the cord 30 around the belt 10 and to attach to the adhesive 28 as desired to accommodate the girth of the wearer 64 within the lower back region K as shown in, for example, FIGS. 9 and 10.

A first reinforcement plate 12 made from, for example, a rigid material, such as plastic, may be positioned, stitched onto and/or otherwise attached to the mesh 18 of the first side piece 20 within the rear connection region A as shown in, for example, FIG. 1. The first reinforcement plate 12 may have perforations 24 formed within the first reinforcement plate 12 to, for example, reduce the weight of the first reinforcement plate 12. A first protrusion 32 may be integrally formed with and extend outwardly from the first reinforcement plate 12. A sleeve 36 may be integrally formed with the first reinforcement plate 12 and may attach to the first protrusion 32. The sleeve 36 and the first protrusion 32 may have hollow interiors to receive, guide and/or otherwise accommodate the cord 30 which may be threaded through the sleeve 36 and/or the first protrusion 32 as shown in FIG. 1, for example.

Specifically, the cord 30 may be inserted in and/or otherwise woven through holes 26 in the first protrusion 32. Accordingly, the cord 30 may extend to, thread into and/or otherwise connect with a corresponding second protrusion 34. In an embodiment, the second protrusion 34 may be integrally formed with and extend from a second reinforcement plate 14 located on the second side piece 22, which may be attached to the mesh 18 thereof. Similar to the first reinforcement plate 12, the second reinforcement plate 14 may be made from a substantially rigid material, such as plastic and/or a composite, and may be stitched onto and/or otherwise attached to the mesh 18 of the second side piece 22. The first reinforcement plate 12 and the second reinforcement plate 14 may be collectively referred to as a back support. In an embodiment, the adhesive strip 28 may extend from the second reinforcement plate 14 on the mesh 18 of the second side piece 22.

Pulling of the tab 40 in either direction C or direction D may extend the cord 30 around the circumference of the support belt 10 that may allow the wearer 64 to affix the tab 40 on the adhesive strip 28 at a location chosen by the wearer 64 to, for example, accommodate the girth of the wearer 64. Further, the belt 64 may be worn tighter around the lower back area K of the wearer 64 which may be desirable in certain strenuous and/or high-load situations. Extension of the cord 30 around the circumference of the support belt 10 may tighten and/or pull the cord 30 through and/or about the holes 26 that may be located on both the first protrusion 32 and/or the second protrusion 34 to generally pull the first reinforcement plate 12 in the direction C toward the second reinforcement plate 14. Likewise, the second reinforcement plate 14 may be pulled in the direction D toward the first reinforcement plate 12 to secure the first side piece 20 to the second side piece 22.

In an embodiment as shown in FIG. 1 for example, the first reinforcement plate 12 may be connected and/or attached with the second reinforcement plate 14 by interweaving the cord 30 through the array of holes 26 at the rear connection area A. Further, the rear connection area A, generally defining the first reinforcement plate 12 and the second reinforcement plate 14, may be aligned with the lower back area K of the wearer 64 to, for example, compress against and/or provide support to the wearer 64. Thus, upon pulling, tightening and/or attachment of the tab 40 on the adhesive strip 28 extending along the circumference of the support belt 10 on the mesh 18, the wearer 64 may compress the first reinforcement plate 12 and/or the second reinforcement plate 14 against the wearer 64. Such compression may assist the wearer 64 in alleviating and/or otherwise avoiding potential sources and/or causes of pain in the lower back area K that may be associated with and/or otherwise caused by the repetitive lifting and/or moving of objects, for example.

To wear the support belt 10, the wearer 64 may, for example, grasp the support belt 10 which may have the cord 30 pre-threaded as shown in FIGS. 1 and 2, for example, and may wrap the first side piece 20 toward the second side piece 22. Accordingly, the wearer may attach the first fastener panel 44 on the first side piece 20 with the corresponding second fastener panel 46 on the second side piece 22 to secure the support belt 10 to itself in the circumferential direction as shown in, for example, FIGS. 1 and 2. One of ordinary skill in the art will appreciate that the spatial configurations and/or orientations described of the various components of the support belt 10, such as, but not limited to, the first reinforcement plate 12 and/or the second reinforcement plate 14, are exemplary in nature and not otherwise intended to depart from the scope and spirit of the invention. For example, other types and/or methods of adhering the tab 40 to the adhesive strip 28 may be used other than Velcro® and/or Velcro-type fasteners, for example. Further, the positioning of panels of fasteners may be interchanged and/or inverted to accommodate attachment of the first side piece 20 to the second side piece 22.

Referring to FIG. 2, the support belt 10 is shown illustrating pulling, sliding and/or other general movement of the tab 40 connected to the cord 30 in a direction F in the front region B. The tab 40 may be interwoven with a loop 42 wrapped around a ring 60 connected to the cord 30. In an embodiment, the wearer may pull the tab 40 in the direction F to attach to the adhesive strip 28 to tighten and/or to loosen the support belt 10 in the circumferential direction. Grommets 38 may extend through, provide structural definition to and/or assist in the attachment of the first reinforcement plate 12 and/or the second reinforcement plate 14 to the mesh 18 stretched across the perimeter 16 of the support belt 10.

In an embodiment, the wearer 64 of the support belt 10 may pull the cord 30 in the direction E and/or F, as shown in FIG. 2, for example, to constrict and/or otherwise reduce the overall circumference of the support belt 10 and/or to accommodate the girth of the wearer 64. For example, the wearer 64 may pull the tab 40 along the adhesive strip 28 on the second side piece 22 to attach to the same. One of ordinary skill in the art will appreciate that the relative orientations and/or placement of the tab 40 that may be connected to the cord 30 are exemplary, and the support belt 10 may be worn inverted and/or the tab 40 may be extended in a direction opposite to directions E and/or F, as shown in FIG. 2 to wrap the cord 30 around in a direction opposite to that shown in FIG. 2. In such a configuration, the cord 30 may also reduce the overall circumference of the support belt 10 to accommodate the girth of the wearer 64.

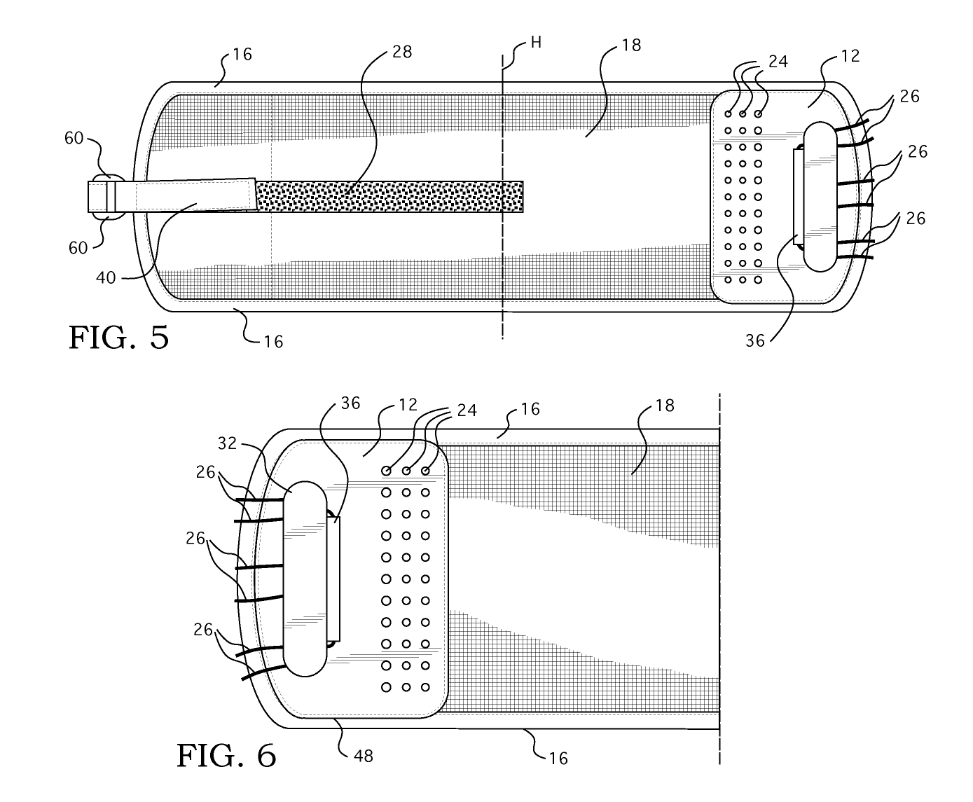

Referring to FIG. 3, the support belt 10 is shown as unfolded and/or otherwise laid-flat such that the second side piece 22 may be positioned adjacent to the first side piece 20. In an embodiment, the support belt 10 may be folded across a central axis G for storage and/or transport as shown in, for example, FIGS. 4 and 5. Further, the support belt 10 may be folded again more, for example, across mid-axes H and/or I after first folding across the central axis “G” to form a more compact form of the support belt 10 as shown in, for example FIG. 6. The second fastener panel 46 may be attached to the mesh 18 of the second side piece 22 to fit with and/or otherwise engage with the corresponding first fastener panel 44 attached to a location on the first side piece 20, as shown in FIG. 3.

Specifically, the second fastener panel 46 may be attached to an exterior-facing surface of the mesh 18 of the second side piece 22 to engage with the first fastener panel 44, which may be attached to the mesh 18 of the first side piece. Accordingly, the second fastener panel 46 may attach to, engage with and/or otherwise combine with the first fastener panel 44 in the front region B as shown in FIG. 2, for example to secure the belt circumferentially around the lower back area K of the wearer 64 as shown in, for example, FIGS. 9 and 10. One of ordinary skill in the art will appreciate that the relative configuration and/or positioning of each the second fastener panel 46 on the second side piece 22 and/or the first fastener panel 44 on the first side piece 20 is exemplary and thus not intended to be limiting to the specific configuration and/or positioning shown in the figures. Alternative suitable configurations may exist that may also accommodate attachment of the second side piece 22 to the first side piece 20 as shown in, for example, FIGS. 1 and 2, to wrap around the lower back area K of the wearer 64 as shown in, for example, FIGS. 9 and 10.

Referring to FIG. 4, the support belt 10 is shown folded onto itself along the central axis “G” as shown in FIG. 3, for example, to expose the second side piece 22. In an embodiment, the support belt 10 may be folded as shown in FIG. 4 to reduce in size and thus accommodate transport and/or storage. As shown in FIG. 4, the tab 40 connected to the cord 30 may be extended across the second side piece 22 lengthwise to span the length of the adhesive strip 28 and attach to the second fastener panel 46. Accordingly, the support belt 10 may be further folded across mid-axis “I” to form a compact configuration as shown in FIG. 6, for example. Guide passages 52 may be positioned between the perforations 24 and the second protrusion 34 on the second reinforcement piece 14 to guide the cord 30 from the of holes 26 across the adhesive strip 28 on the mesh 18 and toward the second fastener panel 46 that may be attached to the mesh 18.

Referring to FIG. 5, the support belt 10 is shown folded over upon itself along the central axis G as shown in FIG. 3 such that the first side piece 20 may be exposed. In an embodiment, the support belt 10 may be folded as shown in FIG. 5 to reduce in size and thus accommodate transport and/or storage. A redundant description of like elements first introduced and discussed in connection to the preceding FIGS. 1-4 is omitted. As shown in FIG. 5, the adhesive strip 28 may extend partially across the mesh 18 of the first side piece 20 to permit the wearer 64 to extend the tab 40 that may be connected to the cord 30 across the front region B, as shown in FIG. 2, for example, to secure the tab 40 to a position on the adhesive strip 38 proportionate to the girth of the wearer 64. In an embodiment, the cord 30 may be inserted into and/or otherwise threaded through the sleeve 36 and the first protrusion 32 and extend through and/or across the holes 26 toward the second protrusion 34 on the second reinforcement piece 14 on the second side piece 22.

Referring to FIG. 6, the support belt 10 is shown folded twice, i.e. first across the central axis G, as shown in FIG. 3, and second across mid-axes H and I to form the compact configuration of the belt as shown in FIG. 6. In the configuration shown in FIG. 6, the first side piece 20 is shown exposed. However, one of ordinary skill in the art will appreciate that the configuration shown in FIG. 6 is exemplary and the support belt 10 may be folded twice over across the central axis G and the mid-axes H and I to expose the second side piece 22, if desired. The support belt 10, in the reduced size configuration shown in FIG. 6, may be stored and/or transported.

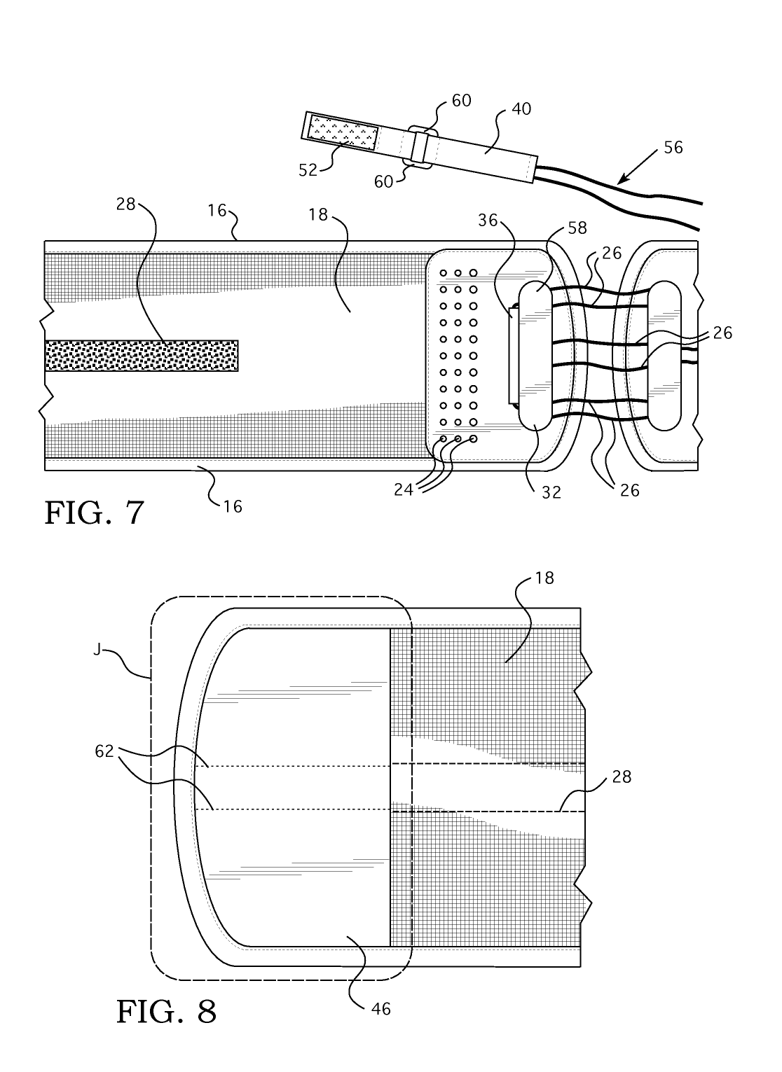

Referring to FIG. 7, in an embodiment, an extension 66 is shown connected to the tab 40 by the ring 50. The wearer 64 may pull and/or otherwise maneuver the extension 66 to position the tab on the adhesive strip 28 for attachment as desired to accommodate the girth of the wearer. The extension 66 may be connected to the tab 40 which may connect with the cord 30 that may be positioned along and/or across either the first side piece 20 or the second side piece 22 for securing the support belt 10 around the lower back area K of a wearer and/or for storage.

Referring to FIG. 8, a close-up view of the second fastener panel 46 is shown positioned at an end J of the second side 22. In an embodiment, the second fastener panel 46 may be positioned on an interior-facing surface of the mesh 18 of the second side 22 to combine with the first side 20 at the front area B as shown in FIG. 2, for example, and/or as described in connection with FIG. 3. Threads 62 may extend length-wise across the second fastener panel 46 to attach the same to the mesh 18.

Referring to FIGS. 9 and 10, a front view and a rear view of the support belt 10 worn by a wearer 64 is shown, respectively. The support belt 10 may provide lower back and/or lumbar support at the lower back region K to reinforce and/or support the wearer 64 as he or she engages in physically demanding and/or strenuous activity. Specifically, the front area B and/or the rear area A of the support belt 10 may be aligned as desired by the wearer 64 to maximize and/or otherwise provide optimal support and/or protection to the wearer 64.

Although the preceding description has been described herein with reference to particular means, materials, and embodiments, the description is not intended to be limited to the particulars disclosed herein; rather, the description extends to all functionally equivalent structures, methods, and uses, such as are within the scope of the appended claims.

We claim:

1. An apparatus comprising:
a belt with a first section and a second section wherein the second section is substantially symmetrical and positioned opposite to the first section and further wherein the first section connects to the second section at a rear connection region;
a reinforcement plate attached to the first section and the second section at the rear connection region wherein the reinforcement plate is made from a substantially rigid material;
an adhesive strip attached to the first section and the second section;
a first fastener panel on the first section and a second fastener panel on the second section positioned to attach to the first fastener to secure the belt around a lower back area of a wearer of the belt; and
a cord interwoven through the reinforcement plate to secure the first section to the second section wherein extension of the cord tightens the belt.

2. The apparatus of claim 1 further comprising:
a perforation in the reinforcement plate to reduce the weight of the reinforcement plate.

3. The apparatus of claim 1 further comprising:
a protrusion extending from the reinforcement plate to direct the cord across the first section and the second section.

4. The apparatus of claim 1 wherein the belt folds to reduce in size.

5. The apparatus of claim 1 wherein the first section and the second section extend from the rear connection region to wrap around the lower back area of the wearer of the belt.

6. The apparatus of claim 1 wherein the cord attaches to the adhesive strip to secure the belt in a tightened position around the lower back area of the wearer of the belt.

7. The apparatus of claim 1 wherein the cord is pulled toward the first section or the second section to tighten the belt.

8. A system comprising:
a belt having a first side and a second side positioned opposite to the first side wherein the first side and the second side each have a perimeter with a mesh stretched across the perimeter;
a plate attached to the mesh of the first side and the second side wherein the plate is positioned adjacent to a lower back of a wearer of the belt to support the back during movement;
a strip attached to the mesh wherein the strip extends across the belt; and
a cord threaded through the plate wherein the cord compresses the belt to secure the belt on the wearer wherein the cord attaches to the strip to retain the belt in a fixed position.

9. The system of claim 8 further comprising:
a grommet extending through the plate.

10. The system of claim 8 further comprising:
a guide passage extending from the plate to direct the cord around the belt.

11. The system of claim 8 further comprising:
a tab connected to the cord allowing for the cord to be grasped by the tab.

12. The system of claim 8 further comprising:
a first panel attached to the mesh on the first side; and
a second panel attached to the mesh on the second side wherein the first panel and the second panel engage to secure the belt around the wearer.

13. The apparatus of claim 8 further comprising:
a first fastener on the first side and a corresponding second fastener on the second side positioned opposite to the first side wherein the first fastener engages with the second fastener to secure the belt around the lower back of the wearer.

14. The apparatus of claim 8 wherein the cord extends either toward the first side or toward the second side.

15. The apparatus of claim 8 wherein the mesh is made from interwoven fibers.

16. A method for securing a belt around a wearer, the method comprising the steps of:
wrapping the belt around a back area of the wearer wherein the belt has a first side section and a second side section substantially identical to and positioned adjacent to the first side section;
attaching a first fastener panel on the first side section to a corresponding second fastener panel on the second side section to secure the belt around the back area of the wearer;
pulling a cord threaded through the first side section and the second side section to tighten the belt around the wearer; and
fixing the cord to an adhesive strip extending across the first side section and the second side section to secure the belt in a fixed position on the wearer.

17. The method of claim 16 further comprising the step of:
directing the cord through guide passages extending from the second side section.

18. The method of claim 16 further comprising the step of:
folding the first side section onto the second side section to reduce a dimension of the belt.

19. The method of claim 16 further comprising the step of:
compressing the belt around the lower back area of the wearer.

20. The method of claim 16 further comprising the step of:
grasping the cord at a tab connected to the cord.
